(19) United States
(12) Patent Application Publication
(10) Pub. No.: US 2013/0236544 A1
(43) Pub. Date: Sep. 12, 2013

(54) STABLE PHARMACEUTICAL COMPOSITIONS OF FESOTERODINE

(71) Applicant: DR. REDDY'S LABORATORIES LTD., Hyderabad (IN)

(72) Inventors: Harshal Prabhakar Bhagwatwar, Hyderabad (IN); Venkateswarlu Vobalaboina, Hyderabad (IN); Pradip Kumar Ghosh, West Midnapur (IN); Srinivasa Rao Gella, Jaggayya Pet (IN); Ramakoti Reddy Ch, Atchampet (IN); Sandip Shivaji Chavhan, Yavatmal (IN); Pratit Premchand Agrawal, Gondia (IN)

(73) Assignee: DR. REDDY'S LABORATORIES LTD., Hyderabad (IN)

(21) Appl. No.: 13/786,900

(22) Filed: Mar. 6, 2013

(30) Foreign Application Priority Data
Mar. 8, 2012 (IN) 881/CHE/2012

(51) Int. Cl. A61K 47/26

(52) U.S. Cl. CPC A61K 47/26
USPC 424/465; 514/546; 264/12

(57) ABSTRACT

The present invention relates to stable pharmaceutical compositions comprising fesoterodine or a pharmaceutically acceptable salt thereof. In particular, the invention relates to pharmaceutical compositions of fesoterodine or a pharmaceutically acceptable salt thereof and a stabilizer. The invention also relates to processes for making such compositions and the methods of using such compositions.

### FIELD OF THE INVENTION

The present invention relates to stable pharmaceutical compositions comprising fesoterodine. In particular, the invention relates to pharmaceutical compositions comprising fesoterodine, at least one sugar or its derivatives, and one or more other pharmaceutically acceptable excipients. The invention also relates to processes for making such compositions and use thereof in treating patients with urinary incontinence. The compositions of the present invention are highly stable and provide the drug release for the intended duration of time to a subject in need thereof.

### BACKGROUND OF THE INVENTION

Fesoterodine is a competitive muscarinic receptor antagonist useful in the treatment of overactive bladder with symptoms of urge urinary incontinence, urgency, and frequency. Overactive bladder is a bladder function disorder resulting in symptoms of urgency, with or without urge incontinence, and usually includes increased urinary frequency and nocturia. The disorder is due to spastic contractions of the detrusor muscle of the bladder, resulting in sustained high bladder pressure and the urgent need to urinate. This can be caused by several reasons, such as traumatic or toxic nerve damage (e.g., abdominal trauma, pelvic trauma or surgery, bladder stones, adverse effects of drugs), neurological diseases (e.g., spinal cord lesions, multiple sclerosis, Parkinson's disease, excessive neurotransmitter release in the bladder) or myogenic instability (e.g., bladder hypertrophy caused by outlet obstruction or urinary tract infection).

In some cases, overactive bladder can be managed without pharmacotherapy, using exercise, pessaries, implants, biofeedback or behavioral therapy. But in most cases, pharmacotherapy is the better option. Antimuscarinic agents have been found to be particularly effective for treating overactive bladder. During normal micturition, acetylcholine released from postganglionic parasympathetic neurons acts on the muscarinic receptors of the detrusor smooth muscle in the bladder to stimulate contractions. Antimuscarinic agents interfere with this action, thus reducing detrusor contractions.

Fesoterodine fumarate has a chemical name isobutyric acid 2-((R)-3-diisopropylammonium-1-phenylpropyl)-4-(hydroxymethyl) phenyl ester hydrogen fumarate. The empirical formula of fesoterodine fumarate is C₃₀H₄₁NO₇ and its molecular weight is 527.66. Fesoterodine fumarate is a white to off-white powder, which is freely soluble in water.

A commercially available product containing fesoterodine fumarate is marketed in USA under the brand name TOVIAZ® tablets and are available in the dosages of 4 mg and 8 mg fesoterodine fumarate. TOVIAZ® is indicated for the treatment of overactive bladder with symptoms of urge urinary incontinence, urgency and frequency.

U.S. Pat. Nos. 6,858,650 and 7,384,980 disclose fesoterodine and its pharmaceutical acceptable salts.

International Publication No. WO 2009/044278 discloses amorphous form of fesoterodine fumarate, its process of preparation and pharmaceutical compositions thereof.

U.S. Pat. No. 7,807,715 and U.S. Application Publication No. 2009/117159 disclose a pharmaceutical granulate comprising fesoterodine or a pharmaceutically acceptable salt thereof and a pharmaceutically acceptable stabilizer, which can be selected from the group consisting of sorbitol, xylitol, polydextrose, isomalt, dextrose and combinations thereof. According to these applications, sugar alcohols are very essential to prepare stable compositions of fesoterodine. The prior art exemplifies the use of sugar alcohols for improving the stability of fesoterodine compositions. It has been mentioned that sugar alcohols especially xylitol and sorbitol are necessary to make stable compositions of fesoterodine, without which stability is difficult to achieve.

International Publication No. WO 2011/117884 discloses pharmaceutical compositions of fesoterodine which does not contain sugar alcohols. The prior art exemplifies without the use of sugar alcohols for achieving stable fesoterodine pharmaceutical compositions.

Fesoterodine may exhibit substantial degradation under stress conditions, e.g., in a humid environment and at increased temperature. It is believed that hydrolyzation and oxidation are among the major mechanisms resulting in degradation. Therefore, it would be desirable to develop novel pharmaceutical compositions comprising fesoterodine that are more stable against degradation over an extended period of time even under stress conditions. Although the prior art teaches incorporating sugar alcohols like xylitol and sorbitol stabilizes the fesoterodine formulation, there remains a need to devise alternative pharmaceutical formulations of fesoterodine by using sugars, which exhibits significant stability against degradation over the storage period. Unlike the prior art, the subject invention favorably influences the stability of fesoterodine formulations with sugar or its derivatives.

Inventors of the present invention have surprisingly found that it is possible to formulate stable pharmaceutical composition of fesoterodine or a pharmaceutically acceptable salt thereof using sugar or its derivatives selected from sucrose, fructose, galactose, lactitol, inositol, erythritol and the like, which provides optimum chemical, physical and polymorphic stability of fesoterodine or a pharmaceutically acceptable salt thereof during the manufacturing process and also during storage. The present invention meets the unfulfilled needs in the prior art by providing stable pharmaceutical compositions comprising fesoterodine or a pharmaceutically acceptable salt thereof.

### SUMMARY OF THE INVENTION

Aspects of the present application relate to stable pharmaceutical compositions comprising fesoterodine or a pharmaceutically acceptable salt thereof and a stabilizer, and processes for preparing the same.

Aspects of the present application relate to stable pharmaceutical compositions comprising fesoterodine or a pharmaceutically acceptable salt thereof and a sugar or its derivatives as stabilizer and processes for preparing the same. Further aspects of the application relate to pharmaceutical compositions containing fesoterodine or a pharmaceutically acceptable salt thereof, and methods of use, treatment, and administration involving the compositions.

In an aspect, there are provided stable pharmaceutical compositions comprising fesoterodine or a pharmaceutically acceptable salt thereof.

In an aspect, there are provided stable film coated pharmaceutical compositions comprising fesoterodine or a pharmaceutically acceptable salt thereof and sugar or its derivatives as stabilizer.

In an aspect, there are provided stable pharmaceutical compositions comprising fesoterodine or a pharmaceutically acceptable salt thereof and sugar or its derivatives selected from mannitol, maltitol, sucrose, fructose, galactose, lactitol, inositol, erythritol and the like.

In an aspect, there are provided stable pharmaceutical compositions comprising fesoterodine or a pharmaceutically acceptable salt thereof, sugar or its derivatives selected from mannitol, maltitol, sucrose, fructose, galactose, lactitol, inositol, erythritol and the like, and at least one is rate controlling polymer.

In an aspect, there are provided stable pharmaceutical compositions comprising fesoterodine or a pharmaceutically acceptable salt thereof, sugar or its derivatives as stabilizer and at least one rate controlling polymer wherein the pharmaceutical compositions further comprise one or more pharmaceutically acceptable excipients.

In an aspect, there are provided stable pharmaceutical compositions comprising fesoterodine or a pharmaceutically acceptable salt thereof and sucrose as a stabilizer, and processes for preparation thereof.

In an aspect, there are provided stable pharmaceutical compositions comprising fesoterodine or a pharmaceutically acceptable salt thereof and fructose as a stabilizer, and processes for preparation thereof.

In an aspect, there are provided processes for the preparation of stable pharmaceutical compositions comprising or a pharmaceutically acceptable salt thereof and sugar or its derivatives selected from mannitol, maltitol, sucrose, fructose, galactose, lactitol, inositol, erythritol and the like.

In an aspect, there are provided processes for the preparation of stable pharmaceutical compositions comprising fesoterodine or a pharmaceutically acceptable salt thereof and sugar or its derivatives selected from mannitol, maltitol, sucrose, fructose, galactose, lactitol, inositol, erythritol and the like, one or more rate controlling polymer and one or more pharmaceutically acceptable excipients.

In an aspect, there are provided processes for the preparation of stable pharmaceutical composition comprising fesoterodine or a pharmaceutically acceptable salt thereof and sugar or its derivatives, wherein methods of preparing said compositions include one or more of processes such as direct compression, wet granulation, dry granulation, solvent evaporation, hot melt granulation, hot melt extrusion, fluid bed granulation, spray drying, and extrusion-spheronization.

In an aspect, there are provided methods of treating overactive bladder with symptoms of urge urinary incontinence, urgency, and frequency using stable pharmaceutical compositions comprising fesoterodine or a pharmaceutically acceptable salt thereof and sugar or its derivatives selected from mannitol, maltitol, sucrose, fructose, galactose, lactitol, inositol, erythritol and the like.

In an aspect, there are provided stable pharmaceutical compositions comprising fesoterodine or a pharmaceutically acceptable salt thereof and sugar or its derivatives, wherein the pharmaceutical compositions are stable for commercially relevant period and provide the desired therapeutic concentration of the active agent for the intended duration.

In an aspect, there are provided stable pharmaceutical compositions comprising fesoterodine or a pharmaceutically acceptable salt thereof and sugar or its derivatives selected from mannitol, maltitol, sucrose, fructose, galactose, lactitol, inositol, erythritol and the like, suitable for once daily administration.

In an aspect, there are provided stable pharmaceutical compositions comprising fesoterodine or a pharmaceutically acceptable salt thereof and sugar or its derivatives selected from mannitol, maltitol, sucrose, fructose, galactose, lactitol, inositol, erythritol and the like, preferable fructose, galactose and sucrose, wherein pharmaceutical compositions can be prepared in the form of tablets, granules, matrix tablets, multilayered tablets, powders, pellets, capsules, minitablets, microcapsules, multiple unit particles, and the like.

In an aspect, there are provided stable pharmaceutical compositions comprising fesoterodine or a pharmaceutically acceptable salt thereof, wherein the polymorphic stability of the fesoterodine or a pharmaceutically acceptable salt thereof is achieved during the preparation of the compositions and also during the shelf-life of the formulations.

In an aspect, there are provided stable pharmaceutical compositions comprising fesoterodine or a pharmaceutically acceptable salt thereof, which are prepared into dosage form to exhibit immediate release, extended release, sustained release, controlled release, modified release and delayed release or combination thereof.

In an aspect, there are provided stable pharmaceutical compositions comprising fesoterodine or a pharmaceutically acceptable salt thereof and sugar or its derivatives, which are prepared into dosage form to exhibit an extended release of the active agent for prolonged duration of time.

In an aspect, there are provided stable pharmaceutical compositions comprising fesoterodine or a pharmaceutically acceptable salt thereof, wherein the percentage release of fesoterodine or a pharmaceutically acceptable salt thereof, is at least about 10% in 4 hours, at least about 20% in 8 hours, at least about 30% after 12 hours, and at least about 60% in 24 hours; when subjected to an in vitro dissolution study or administered in vivo to a subject in need thereof.

In an aspect, there are provided stable pharmaceutical composition comprising fesoterodine or a pharmaceutically acceptable salt thereof and sugar or its derivatives selected from mannitol, maltitol, sucrose, fructose, galactose, lactitol, inositol, erythritol and the like, preferable fructose, galactose and sucrose, wherein fesoterodine or a pharmaceutically acceptable salt thereof is present in amount of about 1 mg to about 50 mg, or about 2 mg to about 8 mg.

In embodiments, the present invention provides pharmaceutical compositions comprising fesoterodine or a pharmaceutically acceptable salt thereof, wherein the said active agent fesoterodine or a pharmaceutically acceptable salt thereof has particle size distributions wherein D₉₀ is about 1 μm to about 500 μm, or about 1 μm to about 100 μm; and D₅₀ is from about 1 μm to about 100 μm, or about 1 μm to about 50 μm.

In embodiments, the present invention provides stable compositions comprising fesoterodine or a pharmaceutically acceptable salt thereof, which are substantially free of degradation impurities during manufacturing and after commercially relevant storage periods.

In embodiments, the present invention provides stable formulations comprising fesoterodine or a pharmaceutically acceptable salt thereof, wherein levels of one or more of the impurities, including process related or degradation related impurities, are present in amounts less than about 5%, or less than about 2%, or less than about 1%, or less than about 0.5% by weight of the labeled fesoterodine content.

In embodiments, the present invention provides stable formulations wherein total drug-related impurities, as determined using high performance liquid chromatography (HPLC), are less than about 5% by weight of the labeled fesoterodine content.

In embodiments, the present invention provides processes for producing stable fesoterodine-containing granules, wherein the granules exhibit losses on drying (LOD) in the range of about 0.1-10%, or about 0.5-5%, or about 1-3%, by weight.

In an aspect, there are provided stable pharmaceutical compositions comprising fesoterodine or a pharmaceutically acceptable salt thereof, and sugar or its derivatives selected from mannitol, maltitol, sucrose, fructose, galactose, lactitol, inositol, erythritol and the like, wherein the pharmaceutical compositions are packaged in a strip or a blister or a HDPE container optionally together with a desiccant and/or oxygen absorbent.

Various polymorphic structures of fesoterodine or a pharmaceutically acceptable salt thereof are known. The present application is applicable to all of these structures.

### DETAILED DESCRIPTION OF THE INVENTION

It has been surprisingly found by the inventors of the present invention that the use of a stabilizer compound such as a sugar or its derivatives to form pharmaceutical compositions of fesoterodine or a pharmaceutically acceptable salt thereof results in improved stability particularly with respect to the levels of impurities, and other drug-related substances that are likely to form during preparation of composition or during storage.

Aspects of the present application provide stable pharmaceutical compositions comprising fesoterodine or a pharmaceutically acceptable salt thereof and a stabilizer, and processes for preparing the same.

Aspects of the present application provide stable pharmaceutical compositions comprising fesoterodine or a pharmaceutically acceptable salt thereof and a sugar or its derivatives as stabilizer and processes for preparing the same.

In an aspect, there are provided stable pharmaceutical compositions comprising fesoterodine or a pharmaceutically acceptable salt thereof and sugar or its derivatives selected from mannitol, maltitol, sucrose, fructose, galactose, lactitol, inositol, erythritol and the like.

In an aspect, there are provided stable pharmaceutical compositions comprising fesoterodine or a pharmaceutically acceptable salt thereof, sugar or its derivatives selected from mannitol, maltitol, sucrose, fructose, galactose, lactitol, inositol, erythritol and the like, and at least one is rate controlling polymer.

The term “fesoterodine” or “fesoterodine or a pharmaceutically acceptable salt thereof” as used herein includes salts, active metabolites, polymorphs, hydrates, solvates, esters, prodrugs, derivatives, isomers or enantiomers. The fesoterodine salt can be a salt of a polybasic acid. Examples may be chosen from the group of polybasic mineral acids, such as e.g. sulfuric acid and phosphoric acid, or of polybasic organic acids. Preferred examples are salts of di- or tricarboxylic acids such as fesoterodine maleate, fesoterodine oxalate, fesoterodine citrate, fesoterodine phthalate, fesoterodine fumarate, fesoterodine succinate, fesoterodine tartrate, fesoterodine malonate, fesoterodine malate, etc. In particular embodiments, the fesoterodine salt may be a partially hydrogenated di- or tricarboxylic acid salt, particularly a salt such as hydrogen fumarate or hydrogen maleate. A particularly preferred salt is fesoterodine hydrogen fumarate.

Fesoterodine or a pharmaceutically acceptable salt thereof, preferably Fesoterodine hydrogen fumarate, or the free base, in the composition may be present in an amount of about 0.5-50 mg, or about 0.5-20 mg, or about 1-16 mg, or about 1-12 mg, or about 1-8 mg, or about 2 mg, or about 4 mg or about 8 mg per dosage unit (based on the content of Fesoterodine or its salt, e.g., Fesoterodine hydrogen fumarate, or free base).

The term “stabilizer” refers to substances, which inhibits, prevents, slows down, or reduces the degradation of Fesoterodine as compared to Fesoterodine in the absence of the substance such as but not limited to mannitol, maltitol, sucrose, fructose, galactose, lactitol, inositol, erythritol and the like. The stabilizer can account for about 1-50% or about 1-30% or about 1-20%, or about 1-10% or about 1-5% or about 30% or about 25% or about 20% or about 15% or about 10% or about 5% of the total composition.

The term “sugar or its derivatives” as used herein includes sugars such as but not limited to fructose, glucose, mannose, galactose, lactose, sucrose, maltose and the like, sugar alcohols, sugar acid, amino sugars and the like.

Thus, in an embodiment, the stable composition of fesoterodine or a pharmaceutically acceptable salt thereof prepared according to the invention with one of more pharmaceutically acceptable excipients was found to have at least 80%, preferably 90% of the original amount of fesoterodine or a pharmaceutically acceptable salt thereof remaining in undegraded form.

The term “excipient” or “pharmaceutically acceptable excipient” means a component of a pharmaceutical product that is not an active ingredient, such as a filler, diluent, carrier, etc. The excipients that are useful in preparing a pharmaceutical composition are generally safe, non-toxic, and neither biologically nor otherwise undesirable, and are acceptable for veterinary use as well as human pharmaceutical use. An “excipient” or a “pharmaceutically acceptable excipient” as used in the specification includes both one and more than one such excipient.

The term “pharmaceutically acceptable excipients” include, for example, any one or more of diluents, binders, rate controlling polymers, lubricants, glidants, disintegrating agents, surfactants, film coating materials, plasticizers, pigments, opacifiers, and coloring agents, and any other materials that are commonly used in solid pharmaceutical dosage form preparations.

Excipients present in pharmaceutical formulations according to the application include diluents such as calcium sulfate, cellulose acetate, dextrates, dextrin, dextrose, fructose, kaolin, lactitol, maltitol, maltodextrin, maltose, polymethacrylates, sodium chloride, sucrose, talc, starches, lactose, mannitol, cellulose derivatives, and the like. Different grades of lactose include, but are not limited to, lactose monohydrate, lactose DT (direct tableting), lactose anhydrous, Flowlac™, Pharmatose™ and others. Different grades of starches include, but are not limited to, maize starch, potato starch, rice starch, wheat starch, pregelatinized starch, Starch 1500, Starch 1500 LM grade (low moisture content grade), fully pregelatinized starch, and others. Various cellulose compounds that can be used include crystalline cellulose, powdered cellulose, and cellulose acetate. Examples of crystalline cellulose products include, but are not limited to, CEOLUS™ KG801, Avicel™ PH101, PH102, PH301, PH302, and PH-F20, microcrystalline cellulose 114, silicified microcrystalline cellulose, and microcrystalline cellulose 112, MICROCELAC™ 100. Other useful diluents include, but are not limited to, carmellose, sugar alcohols such as mannitol, sorbitol, and xylitol, calcium carbonate, magnesium carbonate, sodium carbonate, sodium bicarbonate, light magnesium oxide, heavy magnesium oxide, sodium hydrogen phosphate, calcium sulfate, disodium hydrogen phosphate, basic calcium phosphate, and tribasic calcium phosphate.

Pharmaceutical formulations according to the present invention can also include binders, such as carboxymethylcelluloses, hydroxyethylcelluloses, dextrin, gelatin, maltodextrin, polyethylene oxides, sodium alginate, hydroxypropylcelluloses, hydroxypropyl methylcellulose, polyvinylpyrrolidones or povidone (e.g., PVP-K25, PVP-K29, PVP-K30, and PVP-K90D), powdered acacia, gelatin, guar gum, carbomers (e.g., a Carbopol™ product), methyl celluloses, polymethacrylates, and starches.

Disintegrants can also be present, such as carmellose calcium, carboxymethylstarch sodium, croscarmellose sodium, crospovidone, examples of commercially available crospovidone products including but not being limited to crosslinked povidone, KOLLIDON™ CL, POLYPLASDONE™ XL, XI-10, and INF-10, and low-substituted hydroxypropylcelluloses. Examples of low-substituted hydroxypropylcelluloses include, but are not limited to, low-substituted hydroxypropylcellulose LH11, LH21, LH31, LH22, LH32, LH20, LH30, LH32, and LH33. Other useful disintegrants include sodium starch glycolate (type A or type B), and starches.

Suitable “rate controlling polymers” may include one or more hydrophilic and hydrophobic polymers or mixtures thereof.

Suitable hydrophilic polymers may include one or more of cellulosic polymers/copolymers or its derivatives including methyl cellulose, hydroxypropyl methylcellulose, hydroxyethyl cellulose, hydroxypropyl cellulose, hydroxyethyl methylcellulose, carboxymethylcellulose, sodium carboxymethylcellulose; polyacrylates, methyl acrylates, polyethylene oxides, polyethylene glycols, chitosan, gums, starch derivatives, polyurethanes, galactomannans, polysaccharides, polyalcohols, acrylic acid or acrylamide derivatives and the like.

Suitable hydrophobic polymers include one or more of ethyl cellulose, glycerol palmitostearate, beeswax, glycowax, carnaubawax, hydrogenated vegetable oil, glycerol monostearate, stearylalcohol, glyceryl behenate, polyanhydrides, methyl acrylates and the like.

The stable composition of fesoterodine or a pharmaceutically acceptable salt thereof may be accomplished by homogeneously embedding drug containing rate-controlling hydrophilic polymers, being a soluble, partially soluble or insoluble network of viscous, hydrophilic polymers, held together by physical or chemical entanglements, by ionic or crystalline interactions, by complex formation, by hydrogen bonds or van der Waals forces. The hydrophilic matrix swells upon contact with water, thereby creating a protective gel layer from which the active ingredient is slowly, gradually, continuously released in time either by diffusion through the polymeric network, by erosion of the gel layer, by dissolution of the polymer, or by a combination of these release mechanisms.

The polymers used can also be eroding or non-eroding or combination of both. The polymers, which may be used for bioadhesion, are described below.

Natural polymers include but are not limited to proteins (e.g., hydrophilic proteins), such as pectin, zein, modified zein, casein, gelatin, gluten, serum albumin, or collagen, chitosan, oligosaccharides and polysaccharides such as cellulose, dextrans, tamarind seed polysaccharide, gellan, carrageenan, xanthan gum, gum Arabic; hyaluronic acid, polyhyaluronic acid, alginic acid, sodium alginate.

When the bioadhesive polymer is a synthetic polymer, the synthetic polymer is typically selected from but are not limited to polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, polystyrene, polymers of acrylic and methacrylic esters, polylactides, poly(butyric acid), poly(valeric acid), poly(lactide-co-glycolide), polyanhydrides, polyorthoesters, poly(fumaric acid), poly(maleic acid), and blends and copolymers or mixtures thereof.

Other polymers suitable for use in the invention include, but are not limited to, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobiityl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate)polyethylene, polypropylene, poly(ethylene glycol), poly(ethylene oxide), poly(ethylene terephthalate), polyvinyl acetate), polyvinyl chloride, polystyrene, polyvinyl pyrrolidone, polyvinylphenol, Polylactides, polyglycolides and copolymers thereof, poly(ethylene terephthalate), poly(butyric acid), poly(valeric acid), poly(lactide-co-caprolactone), poly[lactide-co-glycolide], polyanhydrides (e.g., poly(adipic anhydride)), polyorthoesters, blends and copolymers thereof.

Lubricants can also be present as excipients, such as stearic acid, magnesium stearate, calcium stearate, sodium lauryl sulphate, hydrogenated vegetable oil, hydrogenated castor oil, sodium stearyl fumarate, talc, glyceryl behenate, glyceryl monostearate, palmitic acid, carnauba wax, calcium soaps, zinc stearate, polyoxyethylene monostearates, calcium silicate, silicon dioxide, macrogols, and any mixtures thereof.

One or more glidant materials, which improve the flow of powder blends, pellets, or mini-tablets, and minimize dosage form weight variations, can be present as excipients, such as colloidal silicon dioxide, silica derivatives, and talc.

Compositions can also contain wetting agents to improve the wettability of one or both active agents. Various useful surfactants include, but are not limited to, sodium lauryl sulfate, cetrimide, polysorbates such as polysorbate 80, poloxamers such as poloxamer 188 and poloxamer 407, sodium carboxymethylcelluloses, hydrogenated oils, polyoxyethylene glycols, polyoxypropylene glycols, sorbitan fatty acid esters (e.g., SPAN® surfactants), polyoxyethylene sorbitan fatty acid esters (e.g., TWEEN® surfactants), polyglycolized glycerides, available commercially such as GELUCIRE® 40/14, GELUCIRE® 42/12, and GELUCIRE® 50/13, Vitamin E TGPS, and any mixtures of two or more thereof.

Coloring agents can include, but are not limited to, iron oxides, lake of sunset yellow, lake of quinoline yellow, lake of erythrosine, titanium dioxide, FD&C colorants, and the like.

Various solvents can be used in processes of preparing pharmaceutical compositions of the present application including, but not limited to, water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethyl sulphoxide, N,N-dimethylformamide, tetrahydrofuran, and any mixtures thereof.

In embodiments, pharmaceutical compositions of the present application are in the form of film-coated tablets. Useful coating compositions comprise pre-formulated film-coating materials such as OPADRY® products (manufactured by COLORCON), including OPADRY® Blue etc.), other hydrophilic or hydrophobic substances, and mixtures thereof. Useful components for coating include, but are not limited to, plasticizers, antiadherents, opacifiers, solvents, and optionally colorants, lubricants, pigments, antifoam agents, and polishing agents.

In an aspect, a film coating composition contains the following components: polymer, plasticizer, colorant or opacifier, and vehicle. In film coating suspensions, minor quantities of flavors, surfactants, and waxes can be included. In embodiments, polymers used in film coating are cellulose derivatives, such as cellulose ethers, or acrylic polymers and copolymers. High molecular weight polyethylene glycols, polyvinylpyrrolidones, polyvinyl alcohols, and waxy materials can also be used.

Typical cellulose ethers include hydroxyethylcelluloses, hydroxypropylcelluloses, hydroxypropyl methylcellulose, and methylcellulose. Suitable acrylic polymers include synthetic polymers with diverse functionalities. They may be further modified to enhance swelling and permeability by the incorporation of materials such as water soluble cellulose ethers and starches in order to ensure a more complete disintegration or dissolution of the film.

Plasticizers include materials such as polyethylene glycols (PEG), propylene glycols, cetanol, triacetin, citric acid esters such as, for instance, those sold under the trade name CITROFLEX® (Pfizer, New York), phthalic acid esters, dibutyl succinate, castor oil, diacetylated monoglycerides, dibutyl sebacate, diethyl phthalate, glycerin, triethyl citrate, and the like. Suitable plasticizers for use in the coating materials can be categorized into three groups: polyols (e.g., glycerol, propylene glycol, and macrogols), organic esters (e.g., phthalate esters, dibutyl sebacetate, citrate esters, and triacetin), oils or glycerides (e.g., castor oil, acetylated monoglycerides, and fractionated coconut oil).

Pigments, opacifiers such as titanium dioxide, talc, and other additives may also be included in coating compositions. The quantities of the coating applied may vary from about 0.1-20%, or about 0.5-5%, by weight of the total weight of a core composition. In embodiments, a coating is applied either directly onto the cores or onto sub-coated cores, using conventional coating techniques such as, for instance, pan coating or fluidized bed coating methods.

Antiadherents are frequently used in film coating processes to avoid sticking effects during film formation and drying. An example of a useful antiadhesive for this purpose is talc. The antiadherent is frequently present in a film coating in amounts of about 0.5% (w/w) to 15% (w/w), based upon the total weight of the coating.

Suitable colorants/opacifiers can be selected from several groups such as organic dyes and lacquers, inorganic colors, and natural colors.

Film coating dispersions can be prepared using various vehicles, such as water, alcohols, ketones, esters, chlorinated hydrocarbons, and any mixtures thereof.

In embodiments, pharmaceutical compositions of the present invention may comprise a sub-coating, onto which a film coating is provided.

In embodiments, pharmaceutical compositions may be prepared by extrusion and spheronization, or using a melt granulation technique. Compositions may be presented as uncoated, film coated, sugar coated, compression coated, or powder coated forms.

The term “stability” as used herein includes both chemical stability and physical and polymorphic stability. The term ‘stability’ is defined as the ability of a drug substance or drug product to remain within the established specifications to maintain its identity, strength, quality, and purity at least until its expiration date. The term ‘chemical stability’ means the tendency of drug to resist changes or decomposition due to chemical reactions, or due to the effects of oxygen, heat, light, pressure, etc. The term ‘polymorphic stability’ means the tendency of drug to retain its original polymorphic form throughout the product shelf life. The term “physical and polymorphic stability” refers to maintaining the physical and polymorphic form of the active agents, such as crystalline, amorphous, or mixtures thereof, and “chemical stability” refers to maintaining acceptable concentrations of drug-related impurities.

The term ‘shelf life’ is the time that finished products can be stored after manufacturing, during which the defined quality of a specified proportion of the product remains acceptable under expected (or specified) conditions of distribution, storage, and display. The shelf life is established by the manufacturer of a product.

In embodiment, the term “about” refers to quantitative terms, plus or minus 5%, or in another embodiment plus or minus 10%.

The descriptions of excipients are illustrative and are not intended to be exhaustive or limiting. Those skilled in the art will be aware of many other substances that are useful in the practice of the application, and the use of such substances is specifically included in this application.

The selection of appropriate particle sizes of the active agent, as well as of excipients, is within the scope of the application. D₁₀, D₅₀, and D₉₀ values are useful ways for indicating a particle size distribution. D₉₀ is the size value where at least 90 volume percent of the particles have sizes smaller than the value. Likewise, a D₁₀ value refers to 10 volume percent of the particles having sizes smaller than the value. A D₅₀ value refers to 50 volume percent of the particles having sizes smaller than the value, and a D[4,3] value refers to the mean particle size. Methods for determining D₁₀, D₅₀, D₉₀, and D [4,3] include laser diffraction techniques, such as using equipment from Malvern Instruments Ltd., Malvern, Worcestershire, United Kingdom, or from Horiba.

In embodiments, pharmaceutical compositions of the present invention are prepared using active agent fesoterodine or a pharmaceutically acceptable salt thereof, having particle size distributions wherein: D₉₀ is about 1 μm to about 500 μm, or about 1 μm to about 100 μm; and D₅₀ is from about 1 μm to about 100 μm, or about 1 μm to about 50 μm.

In embodiments, pharmaceutical compositions of the present invention are made into suitable pharmaceutical dosage forms. The different pharmaceutical dosage forms include solid oral dosage forms such as, but not limited to, tablets, capsules, and sachets.

In embodiments, the compositions of the present invention can be in the form of multiparticulates such as bilayered minitablets, which can be filled into a capsule.

Pharmaceutical compositions in embodiments of the present application include tablets including an active ingredient, coated with another active ingredient using a film coating and/or compression coating technique.

In an embodiment, the invention further provides a process for preparing the stable pharmaceutical compositions of fesoterodine or a pharmaceutically acceptable salt thereof. The stable pharmaceutical compositions of fesoterodine or a pharmaceutically acceptable salt thereof may be prepared by processes known to the person having ordinary skill in the art of pharmaceutical technology such as direct compression, wet or dry granulation, slugging, hot melt granulation, hot melt extrusion, fluidized bed granulation, extrusion-spheronization, spray drying and solvent evaporation, or a combination of these techniques.

In an embodiment, the stable compositions of fesoterodine or a pharmaceutically acceptable salt thereof is prepared by dry/wet granulating fesoterodine or a pharmaceutically acceptable salt thereof with one of more pharmaceutically acceptable excipients and then optionally mixing the granules with other excipients.

There are no particular limitations on granulation solvents, which may be water or any of various organic solvents, for example, lower alcohols such as methanol and ethanol, ketones such as acetone and methyl ethyl ketone, methylene chloride, or any mixtures thereof.

Further, for granulation, operations such as mixing granulation methods using a planetary mixer, a screw mixer, and the like, high-speed mixing granulation methods using a Henschel mixer, a Super mixer, and the like, extruding granulation methods using a cylindrical granulator, a rotary granulator, a screw extruding granulator, a pellet mill type granulator, and the like, wet high-shear granulation methods, fluidized-bed granulation methods, compression granulation methods, crushing granulation methods, and spraying granulation methods can be used.

After granulation, drying using an oven dryer, a fluidized bed dryer, and the like, crushing, and sieving can be carried out to obtain granules or fine granules for use. Moreover, a granulation solvent may be used when preparing the composition according to the present application.

The term “extragranular” refers to formulation components that are added after or following a granulation step.

Equipment suitable for processing pharmaceutical compositions include any one or more of rapid mixer granulators, planetary mixers, mass mixers, ribbon mixers, fluid bed processors, mechanical sifters, blenders, roller compacters, extrusion-spheronizers, compression machines, capsule filling machines, rotating bowls or coating pans, tray dryers, fluid bed dryers, rotary cone vacuum dryers, and the like, multi-mills, fluid energy mills, ball mills, colloid mills, roller mills, hammer mills, and the like, equipped with a suitable screen wherever required.

The different physicochemical properties of the active agent as well as of excipients are to be considered, as these properties affect processing and formulation aspects. Various important physicochemical properties include, but are not limited to, particle sizes, density (bulk density and tapped density), compressibility index, Hausner's ratio, angle of repose, etc. Particle sizes of active pharmaceutical ingredients can affect the pharmaceutical compositions in numerous ways. For example, content uniformity (CU) of pharmaceutical compositions can be affected by particle sizes and size distributions. Also, particle sizes can play an important role in the dissolution of active agent from the final dosage forms, because of their solubility. Hence, these physicochemical properties not only affect the processes of preparing the pharmaceutical compositions but also affect the performance of the pharmaceutical compositions, both in vitro and in vivo.

In embodiments, tablets have hardness values such as 4-50 kiloponds (KP), or 5-30 KP or 10-20 KP.

In embodiments, tablets have friability less than 5%, or less than 2%, or less than 1%.

In embodiments, pharmaceutical compositions have ‘loss-on-drying’ (LOD) less than about 15%, or less than about 10%, after manufacturing and during their shelf-life.

In embodiments, humidity conditions for the processing areas are controlled, such that the processes are carried out below about 70% relative humidity (RH). Low moisture contents are useful to impart improved drug polymorphic stability to a pharmaceutical composition. Excipients having moisture content less than about 10%, or less than about 5%, are also useful to aid in preventing drug polymorphic conversions.

The term “stability” as used herein includes both chemical stability and physical and polymorphic stability. The term ‘stability’ is defined as the ability of a drug substance or drug product to remain within the established specifications to maintain its identity, strength, quality, and purity at least until its expiration date. The term ‘chemical stability’ means the tendency of drug to resist changes or decomposition due to chemical reactions, or due to the effects of oxygen, heat, light, pressure, etc. The term ‘polymorphic stability’ means the tendency of drug to retain its original polymorphic form throughout the product shelf life. The term “physical and polymorphic stability” refers to maintaining the physical and polymorphic form of the active agents, such as crystalline, amorphous, or mixtures thereof, and “chemical stability” refers to maintaining acceptable concentrations of drug-related impurities.

The term ‘shelf life’ is the time that finished products can be stored after manufacturing, during which the defined quality of a specified proportion of the product remains acceptable under expected (or specified) conditions of distribution, storage, and display. The shelf life is established by the manufacturer of a product.

In embodiment, the term “about” refers to quantitative terms, plus or minus 5%, or in another embodiment plus or minus 10%.

In embodiments, pharmaceutical compositions of the present invention are intended for oral administration to a subject in need thereof.

In an aspect, the present invention provides methods of prophylaxis, amelioration, or treating diseases and/or disorders, by administering a therapeutically effective amount of pharmaceutical compositions to subjects in need thereof.

The term ‘related substances’ or ‘impurities’ mean the degradation impurities or active ingredient process related impurities of drug materials.

Fesoterodine or a pharmaceutically acceptable salt thereof, related impurities include the following:

Impurity 1: 3-[(1R)-3-[bis(1-methylethyl)amino]-1-phenylpropyl]-4-hydroxylbenzenemethanol

Impurity 2: Propanoic acid, 2-methyl-2-[(1R)-3-[bis(1-methylethyl)amino]-1-phenylpropyl]-4-methylphenyl ester

Impurity 3: Propanoic acid, 2-methyl-,2-[3-[bis(1-methylethyl)amino]-1-phenylpropyl]-4-[(2-methyl-1-oxopropoxy)methyl]phenyl ester

Impurity 4: (S-fesoterodine): Propanoic acid, 2-methyl-2-[(1S)-3-[bis(1-methylethyl)amino]-1-phenylpropyl]-4-(hydroxymethyl)phenyl ester (2E)-2-butenedioate (1:1) (salt)

Impurity 5: Isobutyric acid 2-(3-diisopropylamino-1-phenyl-propyl)-4-[2-(3-diisopropyl amino-1-phenyl-propyl)-4-hydroxymethyl-phenoxymethyl]phenyl ester

In embodiments, the present invention provides stable pharmaceutical compositions comprising fesoterodine or a pharmaceutically acceptable salt thereof and sugar or its derivatives as stablizer. In an aspect, the present application provide stable pharmaceutical compositions for oral administration comprising fesoterodine or a pharmaceutically acceptable salt thereof and sugar or its derivatives as stabilizers selected from mannitol, maltitol, sucrose, fructose, galactose, lactitol, inositol, erythritol and the like, preferably fructose or sucrose together with one or more pharmaceutically acceptable excipients and optionally film coating. The application also provides processes for preparing pharmaceutical compositions comprising fesoterodine or a pharmaceutically acceptable salt thereof, and methods of use, treatment, and administration involving the pharmaceutical compositions.

In one embodiment, the present invention provides pharmaceutical compositions in the form of a solid oral dosage forms comprising fesoterodine or a pharmaceutically acceptable salt thereof, sucrose as stabilizer, and one or more pharmaceutically acceptable excipients, optionally wherein the solid dosage form is coated.

In one embodiment, the present invention provides pharmaceutical compositions in the form of a solid oral dosage forms comprising fesoterodine or a pharmaceutically acceptable salt thereof, fructose as stabilizer, and one or more pharmaceutically acceptable excipients, optionally wherein the solid dosage form is coated.

In one embodiment, the present invention provides processes for the preparation of a stable pharmaceutical compositions comprising fesoterodine or a pharmaceutically acceptable salt thereof and sugar or its derivatives selected from mannitol, maltitol, sucrose, fructose, galactose, lactitol, inositol, erythritol and the like, wherein methods of preparing said composition includes direct compression, wet granulation, dry granulation, solvent evaporation, hot melt granulation, hot melt extrusion, fluid bed granulation, spray drying, extrusion-spheronization.

In one embodiment, the present invention provides a process for preparing of a stable pharmaceutical compositions comprising fesoterodine or a pharmaceutically acceptable salt thereof, sugar or its derivatives selected from mannitol, maltitol, sucrose, fructose, galactose, lactitol, inositol, erythritol and the like, preferable fructose and sucrose, and at least one pharmaceutically acceptable excipient, optionally a film coating on the said pharmaceutical composition; wherein the said pharmaceutical compositions are prepared by wet granulation as given below:

i) Dry mixing of fesoterodine or a pharmaceutically acceptable salt thereof, sucrose and/or at least one pharmaceutically acceptable excipient;

ii) Preparation of granulating fluid, optionally containing a binder;

iii) Granulating step i) with step ii) and drying the wet granules to obtain the desired sizes; and

iv) Optionally milling of dried granules followed by mixing with at least one pharmaceutical excipient and lubricant.

In embodiments, the present invention provides a process for preparing of a stable pharmaceutical compositions comprising fesoterodine or a pharmaceutically acceptable salt thereof, sugar or its derivatives selected from mannitol, maltitol, sucrose, fructose, galactose, lactitol, inositol, erythritol and the like, and at least one pharmaceutically acceptable excipient, optionally a film coating on the said pharmaceutical composition; wherein the said pharmaceutical compositions are prepared by wet granulation process as given below:

i) Dry mixing of at least two pharmaceutically acceptable excipients;

ii) Preparation of granulating fluid containing fesoterodine or a pharmaceutically acceptable salt thereof, sucrose and optionally a binder;

iii) Granulating step i) with step ii) and drying the wet granules; and

iv) Optionally milling of dried granules followed by mixing with at least one pharmaceutically acceptable excipient and lubricant.

In embodiments, the present invention provides a process for preparing a stable pharmaceutical compositions comprising fesoterodine or a pharmaceutically acceptable salt thereof, sugar or its derivatives selected from mannitol, maltitol, sucrose, fructose, galactose, lactitol, inositol, erythritol and the like, and at least one pharmaceutically acceptable excipient, optionally a film coating on the said pharmaceutical compositions; wherein the said pharmaceutical compositions are prepared by direct compression process as given below:

i) Dry mixing of fesoterodine or a pharmaceutically acceptable salt thereof, sugar or its derivatives selected from mannitol, maltitol, sucrose, fructose, galactose, lactitol, inositol, erythritol and the like, and least one pharmaceutically acceptable excipient;

ii) adding at least one pharmaceutical excipient and lubricant to step (i) and making into a suitable dosage form.

In embodiments, the present invention provides stable pharmaceutical compositions comprising fesoterodine or a pharmaceutically acceptable salt thereof and sugar or its derivatives selected from mannitol, maltitol, sucrose, fructose, galactose, lactitol, inositol, erythritol and the like, preferable fructose, galactose and sucrose, wherein polymorphic stability of the fesoterodine or a pharmaceutically acceptable salt thereof, is achieved during the preparation of the compositions and also during their shelf-life.

In embodiments, the present application provide methods of treatment using a stable pharmaceutical compositions comprising fesoterodine or a pharmaceutically acceptable salt thereof and sugar or its derivatives selected from mannitol, maltitol, sucrose, fructose, galactose, lactitol, inositol, erythritol and the like.

In embodiments, the present application provide methods of treating patients suffering from overactive bladder that may have symptoms such as urinary incontinence, urinary urge incontinence, urinary urgency and/or urinary frequency by administering a unit dosage form of the fesoterodine compositions described herein.

In embodiments, tablets can be formed in any shapes and sizes, such as round, elongated, capsule-shaped, etc. In embodiments, tablets can be embossed or debossed. To form tablets, compression punches can be coated or uncoated punches and plain, concave, or convex in shape.

In embodiments, the present invention provides multiple layered tablets comprising fesoterodine or a pharmaceutically acceptable salt thereof, sugar or its derivatives selected from mannitol, maltitol, sucrose, fructose, galactose, lactitol, inositol, erythritol and the like, preferable fructose, galactose and sucrose, and at least one pharmaceutically acceptable excipient and optionally the said multiple layered tablets is film coated.

In embodiments, the present invention provides pharmaceutical compositions in the form of solid oral dosage forms comprising fesoterodine or a pharmaceutically acceptable salt thereof, sugar or its derivatives selected from mannitol, maltitol, sucrose, fructose, galactose, lactitol, inositol, erythritol and the like, and one or more pharmaceutically acceptable excipients, wherein the solid dosage forms are in the form of inlay tablets.

In embodiments, the present invention provides pharmaceutical compositions in the form of solid oral dosage forms comprising fesoterodine or a pharmaceutically acceptable salt thereof, sugar or its derivatives selected from mannitol, maltitol, sucrose, fructose, galactose, lactitol, inositol, erythritol and the like, and one or more pharmaceutically acceptable excipients, wherein the solid dosage forms are in the form of tablet-in-tablet.

In embodiments, the present invention provides pharmaceutical compositions that are stable for commercially relevant periods and provide desired therapeutic concentrations of the active agents for the intended duration.

In embodiments, the present invention provides stable pharmaceutical compositions comprising fesoterodine or a pharmaceutically acceptable salt thereof and sugar or its derivatives selected from mannitol, maltitol, sucrose, fructose, galactose, lactitol, inositol, erythritol and the like, wherein fesoterodine or a pharmaceutically acceptable salt thereof are chemically stable during the preparation of the formulations and also during their shelf-life.

In embodiments, the present invention provides solid dosage forms comprising fesoterodine or a pharmaceutically acceptable salt thereof and sugar or its derivatives selected from mannitol, maltitol, sucrose, fructose, galactose, lactitol, inositol, erythritol and the like, wherein the chemical stability and/or polymorphic stability of fesoterodine or a pharmaceutically acceptable salt thereof, are maintained during storage at 40° C. and 75% RH for 1 month, 3 months, or for 6 months.

In embodiments, solid dosage forms of the present invention comprising fesoterodine or a pharmaceutically acceptable salt thereof and sugar or its derivatives selected from mannitol, maltitol, sucrose, fructose, galactose, lactitol, inositol, erythritol and the like, contain any one or more of the impurity 1, impurity 3 and impurity 5, each in amounts less than about 5%, and their total in amounts less than about 8%, during storage at 40° C. and 75% RH for 3 months, or at 30° C. and 65% RH for 6 months, or at 25° C. and 60% RH for about 12 months.

Drug-related impurity contents are expressed in this application as percentages of the label content of the respective drug.

The dosage forms can be subjected to in vitro dissolution testing, such as according to Test 711 “Dissolution” in United States Pharmacopeia 29, United States Pharmacopeial Convention, Inc., Rockville, Md., 2005 (“USP”), to determine the rate at which the active agents are released from the dosage forms, and content of active agents can be determined in dissolution media using techniques such as high performance liquid chromatography (HPLC).

In embodiments, the present invention provides solid dosage forms, comprising fesoterodine or a pharmaceutically acceptable salt thereof and sugar or its derivatives selected from mannitol, maltitol, sucrose, fructose, galactose, lactitol, inositol, erythritol and the like, wherein the release of fesoterodine or a pharmaceutically acceptable salt thereof is at least 20% of fesoterodine is released in 1 hour, at least 40% of fesoterodine is released in 4 hours, at least 60% fesoterodine is released after 8 hours, and at least 70% fesoterodine is released in 16 hours when tested in USP type 2 (paddle) apparatus with 900 mL of phosphate buffer (pH 6.8) as the dissolution medium at 37° C., at 75 rμm paddle speed.

In embodiments, the present invention includes use of tamper-resistant or tamper-evident packages, and/or thermo-insulated packages, for pharmaceutical dosage forms comprising fesoterodine or a pharmaceutically acceptable salt thereof. The pharmaceutical compositions may be packaged into blisters, strips, or containers such as plastic, glass, or metal containers. The packaging materials such as containers including closures, composed of polyethylene and/or polypropylene and/or glass, and blisters or strips composed of aluminum or high-density polypropylene, or polyvinyl chloride, or polyvinyl chloride coated with polyvinylidene dichloride, generally termed PVC/PVDC. The package or packaging material optionally may contain one or more oxygen absorbents or desiccants. In embodiments, pharmaceutical compositions of the present invention can be packaged into thermo-insulated packages wherein aluminum blisters or HDPE bottle containers containing a formulation are packed into suitable thermo-insulated devices such as a thermocol or an expanded polystyrene package. In embodiments, the container containing the composition is double-walled, wherein a hollow space between the walls is filled with air that acts as an insulator. In embodiments, either one or both of the closure and base foils used to make blisters containing a pharmaceutical compositions, contains one or more layers of a desiccant.

In embodiments, compositions of the present invention are highly stable chemically and also exhibit appreciable physical and polymorphic stability during the preparation of the compositions and also during their shelf-life.

The following examples further describe certain specific aspects and embodiments of the application. These examples are provided solely for the purpose of illustration, and should not be construed as limiting the scope of the disclosure in any manner.

### EXAMPLES

### Examples 1 to 3

| | | Example 1 | Example 2 | Example 3 |
|---|---|---|---|---|
| S. No. | Name of the ingredient | mg/tab | mg/tab | mg/tab |
| 1. | Fesoterodine Fumarate | 8.0 | 8.0 | 8.0 |
| 2. | Mannitol | 72.0 | — | — |
| 3. | Maltotol | — | 72.0 | — |
| 4. | Sucrose | — | — | 72.0 |
| 5. | Purified Water* | q.s. | q.s. | q.s. |
| 6. | Microcelac 100 | 77.5 | 77.5 | 92.5 |
| 7. | Hypromellose K4M | 24.0 | 24.0 | 24.0 |
| 8. | Hypromellose K100M | 120.0 | 120.0 | 120.0 |
| 9. | Talc | 8.5 | 8.5 | 8.5 |
| 10. | Glyceryl Behenate | 10.0 | 10.0 | 10.0 |
| 11. | Opadry Blue | 15.0 | 15.0 | 15.0 |

*lost during processing

### Manufacturing Procedure:

1. Fesoterodine Fumarate and Mannitol (Example 1)/Maltitol (Example 2)/Sucrose (Example 3) is granulated with purified Water.
2. The granules are dried in drier till LOD of not more than 1.0% w/w is achieved.
3. The dried granules are milled using Quadro mill, 40G screen.
4. The granules are blended with Microcelac 100, HPMC K4M, HPMC K100M CR, Talc and Glyceryl Behenate in double cone blender.
5. The lubricated blend is compressed into tablets using 13.0×6.5 mm, Oval shape, standard concave punches.
6. The tablets are coated using Opadry II Blue.

### Examples 4 to 6

| | | Example 4 | Example 5 | Example 6 |
|---|---|---|---|---|
| S. No. | Name of the ingredient | mg/tab | mg/tab | mg/tab |
| 1. | Fesoterodine Fumarate | 8.00 | 8.00 | 8.00 |
| 2. | Mannitol | 72.0 | — | — |
| 3. | Maltitol | — | 72.0 | — |
| 4. | Sucrose | — | — | 72.0 |
| 5. | Microcelac 100 | 92.5 | 92.5 | 92.5 |
| 6. | Hypromellose K4M | 24.0 | 24.0 | 24.0 |
| 7. | Hypromellose K100M | 120.0 | 120.0 | 120.0 |
| 8. | Talc | 8.5 | 8.5 | 8.5 |
| 9. | Glyceryl Behenate | 10.0 | 10.0 | 10.0 |
| 10. | Opadry Blue | 15.0 | 15.0 | — |

### Manufacturing Procedure:

1. Fesoterodine fumarate is blended with Mannitol (Example 4)/Maltitol (Example 5)/sucrose (Example 6), Microcelac 100, HPMC K4M, HPMC K100M CR, Talc and Glyceryl Behenate in double cone blender.
2. The lubricated blend is compressed into tablets using 13.0×6.5 mm, Oval shape, standard concave punches.
3. The tablets are coated using Opadry II Blue.

### Example 5

| S. No. | Name of the ingredient | mg/tab |
|---|---|---|
| 1. | Fesoterodine Fumarate | 4.0 |
| 2. | Fructose | 36.0 |
| 3. | Purified Water* | q.s. |
| 4. | Microcellac 100 | 136.5 |
| 5. | Hypromellose K4M | 70.0 |
| 6. | Hypromellose K100M | 70.0 |
| 7. | Talc | 8.5 |
| 8. | Glyceryl Behenate | 10.0 |
| 9. | Opadry Blue | 15.0 |

*lost during processing

### Manufacturing Procedure:

1. Fructose and Fesoterodine Fumarate is granulated with Purified Water.
2. The granules are dried in drier till LOD of NMT 1.0% w/w is achieved.
3. The dried granules are milled using Quadro mill, 40G screen.
4. The granules are blended with Microcellac 100, hypromellose K4M, Hypromellose K100M, Talc and Glyceryl Behenate in double cone blender.
5. The lubricated blend is compressed into tablets using 13.0×6.5 mm, Oval shape, standard concave punches.
6. The tablets are coated using Opadry II Blue.

### Example 6

### Dissolution Studies

The results of the dissolution study show that the release of fesoterodine from the compositions of the invention is comparable to TOVIAZ®. The results are shown in table 1.

TABLE 1

| Time | Cumulative Percent of Fesoterodine Fumarate Released | |
|---|---|---|
| (In hours) | TOVIAZ ® 8 mg tablets | Example 3 |
| 1 | 20 | 19 |
| 4 | 52 | 54 |
| 8 | 76 | 78 |
| 16 | 94 | 94 |

### Example 7

### Stability Testing

Impurity analysis results of TOVIAZ® 8 mg tablets are shown in the table-2 below. Values are percentages of respective label drug content for the formulation.

TABLE 2

| Impurity | Initial | 40° C./75% RH -1 Month |
|---|---|---|
| Impurity 1 | 0.32 | 0.85 |
| Impurity 3 | 0.21 | 0.37 |
| Impurity 5 | 0.04 | 0.14 |
| Maximum unknown impurity | 0.16 | 0.13 |
| Total impurities | 0.87 | 1.74 |

Similar study was conducted on the compositions of the present invention. Tablets of Example 1, 2, 3 and 5, were stored in closed HDPE bottles at 40° C. and 75% RH for 1 month. The initial and stored samples were analyzed for impurity content. The results are shown in Table 3.

TABLE 3

| | Storage Condition | | | |
|---|---|---|---|---|
| | Example 1 | | Example 2 | |
| | RT | 40° C./75% RH | RT | 40° C./75% RH |
| | Pack Details | | | |
| | | HDPE bottle | | HDPE bottle |
| | | with canister | | with canister |
| Duration | Initial | 1 Month | Initial | 1 Month |
| Impurity 1 | 3.162 | 10.548 | 2.3 | 3.389 |
| Impurity 3 | 0.674 | 3.267 | 0.445 | 1.121 |
| Impurity 5 | 0.027 | 0.016 | 0.029 | 0.035 |
| Max Unknown | 0.113 | 1.307 | 0.168 | 0.633 |
| Total | 4.265 | 17.192 | 3.335 | 6.971 |
| | Storage Condition | | | |
| | Example 3 | | Example 5 | |
| | RT | 40° C./75% RH | RT | 40° C./75% RH |
| | Pack Details | | | |
| | | HDPE bottle | | HDPE bottle |
| | | with canister | | with canister |
| Duration | Initial | 1 Month | Initial | 1 Month |
| Impurity 1 | 0.788 | 1.627 | 0.12 | 0.23 |
| Impurity 3 | 0.223 | 0.387 | 0.12 | 0.17 |
| Impurity 5 | 0.030 | 0.024 | 0.03 | 0.03 |
| Max Unknown | 0.120 | 0.218 | 0.03 | 0.06 |
| Total | 1.299 | 2.838 | 0.43 | 0.82 |

The results show that fesoterodine formulations having sucrose or fructose as stabilizers have low impurities under both at initial and under stability conditions.

We claim:

1. A pharmaceutical composition comprising fesoterodine or a pharmaceutically acceptable salt thereof, a stabilizer and one or more pharmaceutically acceptable excipients.

2. The pharmaceutical composition according to claim 1, wherein said stabilizer is sugar or its derivative.

3. The pharmaceutical composition according to claim 2, wherein said stabilizer is selected one or more from mannitol, maltitol, sucrose, fructose, galactose, lactitol, inositol, and erythritol.

4. The pharmaceutical composition according to claim 1, wherein the stabilizer is sugar.

5. The pharmaceutical composition according to claim 4, wherein the sugar is selected one or more form glucose, mannose, galactose, fructose, lactose, sucrose and maltose.

6. The pharmaceutical composition according to claim 1, wherein the amount of stabilizer is about 1% w/w to about 50% w/w of the composition.

7. The pharmaceutical composition according to claim 1, wherein fesoterodine or a pharmaceutically acceptable salt thereof is fesoterodine hydrogen fumarate.

8. The pharmaceutical composition according to claim 1, wherein fesoterodine or a pharmaceutically acceptable salt thereof is present in amount of about 1 mg to about 50 mg.

9. The pharmaceutical composition according to claim 1, wherein one or more pharmaceutically acceptable excipients selected from diluents, binders, rate controlling polymers, lubricants, disintegranting agents, glidants, film coating materials, plasticizers, pigments, opacifiers, and coloring agents.

10. A pharmaceutical composition comprising:
(a) about 0.1-about 10.0% w/w of fesoterodine hydrogen fumarate;
(b) about 5-about 30% sugar
(b) about 1.0-about 10% w/w of binder;
(c) about 5-about 60% w/w of diluent;
(d) about 1-about 60% w/w of rate controlling polymer; and
(e) about 0.1-about 10% w/w of one or more of glidant, and lubricant.

11. The pharmaceutical composition according to claim 1, wherein the pharmaceutical composition is prepared by wet granulation, dry granulation, solvent evaporation, hot melt granulation, hot melt extrusion, fluid bed granulation, spray drying, and extrusion-spheronization process.

12. The process of preparing a pharmaceutical composition according to claim 11, wherein the process is wet granulation comprising the steps of:
a) Granulating fesoterodine, sugar and optionally at least one pharmaceutically acceptable excipient with water;
b). drying the wet granules;
c). Optionally milling of granules to get the desired size;
d). mixing the dried granules with at least one pharmaceutically acceptable excipient; and
e) processing into suitable pharmaceutical composition.

13. The pharmaceutical according to claim 12, wherein the pharmaceutical composition is tablets, granules, matrix tablets, multilayered tablets, powders, pellets, capsules, minitablets, microcapsules, multiple unit particles.

14. A method of treating a patient suffering from overactive bladder by administering a therapeutically effective amount of a pharmaceutical composition according to claim 1.
